(19) United States
(12) Patent Application Publication
(10) Pub. No.: US 2020/0078478 A1
(43) Pub. Date: Mar. 12, 2020

(54) Structural molecule of peptide derivative for PSMA-targeting radiotherapy diagnosis and treatment

(71) Applicants: Ming-Hsin Li, Taoyuan City (TW); Ming-Wei Chen, Taoyuan (TW); Shin-Min Wang, Taoyuan (TW); Shih-Wei Lo, Taoyuan (TW); Chun-Fang Feng, Taoyuan (TW); Cheng-Hui Chuang, Taoyuan (TW); Sheng-Nan Lo, Taoyuan (TW)

(72) Inventors: Ming-Hsin Li, Taoyuan City (TW); Ming-Wei Chen, Taoyuan (TW); Shin-Min Wang, Taoyuan (TW); Shih-Wei Lo, Taoyuan (TW); Chun-Fang Feng, Taoyuan (TW); Cheng-Hui Chuang, Taoyuan (TW); Sheng-Nan Lo, Taoyuan (TW)

(21) Appl. No.: 16/125,787

(22) Filed: Sep. 10, 2018

(51) Int. Cl. A61K 51/04

(52) U.S. Cl. CPC A61K 51/0497

(57) ABSTRACT

A PSMA targeting peptide derivative for radiotherapy, which is a structural molecule developed for diagnosis or treatment of prostate cancer, as prostate-specific membrane antigen (PSMA) is a protein present on the surface of healthy prostate cells, which is often at a high level of expression on the surface of prostate cancer cells, and the molecular composition of PSMA inhibitor is mainly composed of glutamic acid, urea and lysine, in addition to the linker of the present invention, PSMA inhibitor can be combined with a chelating agent and truncated Evans Blue, which can be labeled with radionuclides Ga-67, Ga-68, In-111, Lu-177, Cu-64 or Y-90, used for image analysis and analysis of human prostate cancer tumor pattern as a new PSMA targeting peptide receptor radionuclide therapy (PRRT), and which has a longer half-life in vivo and is featured by specific binding of PSMA for radiotherapy diagnosis or treatment.

### BACKGROUND OF THE INVENTION

### 1. Field of the Invention

The present invention relates to a structural molecule of peptide derivative for PSMA (prostate-specific membrane antigen, PSMA) targeting radiotherapy, and more particularly to a peptide derivative having specific binding to radioactivity for PSMA-targeting and having a long half-life in vivo.

### 2. Description of Related Art

According to statistics on global incidence, mortality and prevalence data provided by the World Health Organization (WHO) International Agency for Research on Cancer (IARC) GLOBOCAN 2012, prostate cancer ranks fourth in common cancers regardless of gender, and ranks second in common cancers in men, about 1.1 million men worldwide has been diagnosed with prostate cancer. Clinical non-invasive diagnostic methods for prostate cancer include digital rectal examination, rectal ultrasound, PSA (prostate-specific membrane antigen) detection, CT (computed tomography), MRI (magnetic resonance imaging), and radionuclide bone angiography. These methods have different shortcomings as described below:

Traditional contrast imaging for prostate cancer relies mainly on rectal ultrasound, CT, and MRI. there is no specificity for prostate cancer with these diagnostic methods, for example, clinically, prostate biopsy prostate cancer can be guided by transrectal ultrasound (TURS). However, the current false negative rate of the TURS six-point system puncture method is about 30%, and there are serious complications.

CT examination cannot distinguish between cancerous tissue and benign proliferative tissue, so it is not clear whether prostate cancer is present. MRI soft tissue has a higher resolution and is superior to CT and ultrasound in the diagnosis of prostate lesions, which can effectively identify prostate cancer and benign prostatic hyperplasia. However, prostate cancer is prone to lymph node metastasis and distal bone metastasis, which reduces the effectiveness of MRI in the diagnosis and staging of prostate cancer.

Tc-99m-MDP bone angiography can be compared with X-ray examination. It can be found 3 to 6 months ahead of time. It is helpful to determine the accurate clinical stage of prostate cancer, although its sensitivity is high but its specificity is low. Clinically, approximately 50% of patients with prostate cancer have a bone metastasis that die within 30 to 35 months after diagnosis. Therefore, the current imaging and sectioning methods have been difficult to meet the clinical requirements for early diagnosis and accurate staging of prostate cancer.

Blood PSA index examination uses blood PSA index as a tumor biomarker for prostate cancer, currently, used as sensitive indicators in diagnosis of prostate cancer. However, when the blood PSA index is 4 to 10 μg/L, it is difficult to diagnose prostate cancer, and the blood PSA index can't reflect the characteristics of clinicopathological sections, and which can't effectively distinguish the local lesions or distant metastasis for lack of specificity.

The main technical contents of radiopharmaceutical molecular angiography include:

F-18-NaF bone angiography: clinical studies indicate that Tc-99m-MDP bone angiography has a sensitivity of 50.8% and a specificity of 82% for prostate cancer bone metastasis. In contrast, F-18-NaF PET/CT angiography has a sensitivity of 93% and a specificity of 54%, and it has a higher spatial resolution, and can perform CT anatomical localization and three-dimensional imaging, which is expected to be an early discovery in clinical imaging methods for prostate cancer bone metastasis. Although F-18-NaF can find more metastases, it does not change the clinical treatment plan.

F-18-FDG angiography: F-18-FDG PET/CT can provide effective clinical data for early diagnosis, staging, optimization, and prognosis evaluation of prostate cancer. However, similar to normal cells, prostate cancer glut expression level is low, it is difficult to distinguish benign lesions, and F-18-FDG is mainly excreted by the urinary system, which will interfere with the diagnosis of prostate cancer, resulting in F-18-FDG has a low detection rate for prostate cancer with limited diagnostic value.

C-11-Choline angiography: C-11-Choline prostate cancer has a significant contrast advantage compared with F-18-NaF. C-11-Choline can be concentrated in tumor cells and retained in cells after being phosphorylated in tumor cells. Sutinen et al's C-11-Choline PET study showed that although the bladder site also has contrast agent concentration, the concentration is low and the prostate can be determined by its position and morphology. A retrospective study of 100 patients with prostate cancer by Picchio et al confirmed that C-11-Choline PET has a better diagnostic power for prostate cancer than F-18-FDG, with a positive rate of 47%, while F-18-NaF is only 27%. The sensitivity of N-[F-18] (F-18-fluoro-methylcholine, F-18-FECH) is 84.7%, and the specificity is 91.1%, indicating that the sensitivity and specificity of tumor diagnosis on the prostate with C-11/F-18-FECH are significantly better than Tc-99m-MDP and F-18-NaF.

Yamaguchi (Eur J Nucl Med Mol Imaging. 2005) and other scholars compared the sensitivity of C-11-CholinePET with MRI to locate primary prostate cancer, and found that C-11-CholinePET sensitivity is close to 100%, while MRI is only 60%. However, C-11-Choline angiography is not effective in identifying primary prostate cancer. F-18-FACBC (anti-1-amino-3-F-18-fluorocyclobutane-1-carboxylic acid) angiography: F-18-FACBC is a matrix of L-type amino acid transporter 1 and alanine, serine and cysteine transporter, which is almost free of renal excretion, so the pelvis can be clearly developed and can be used for prostate cancer detection. Schuster et al reported the results of F-18-FACBC PET/CT examination in nearly 100 patients with suspected recurrent prostate cancer. The positive detection rate of primary prostate cancer is 74%, and the positive detection rate of metastatic tumor is 96%. F-18-FACBC angiography can detect lymph node metastasis that cannot be detected by F-18-FDG, and the effect is significantly better than CT or PET alone. Schuster et al reviewed 598 cases of F-18-FACBC multi-center joint angiography showed that the probe was metabolized faster in vivo and better imaging 30 min after injection. At present, F-18-FACBC has been in phase III clinical trial (NCT01666808) in the mainland China.

F-18-DHT (16β-F-18-fluoro-5-dihydrotestosterone) androgen receptor imaging: in the prostate tissue, DHT is The main androgen is 5 times more concentrated than testosterone and 10 times more potent than the androgen receptor. F-18-DHT has a high ratio of prostate to soft tissue radioactivity and is expected to be used in the diagnosis, staging, prognosis and evaluation of hormone therapy effects of prostate cancer. A comparison of F-18-DHT and F-18-FDG in a patient with 7 cases of prostate cancer showed that F-18-DHT metabolized is faster in the body and the uptake of the lesion increased with time. Among the 59 lesions detected, 46 lesions were found in F-18-DHT (detection rate 78%), and 57 lesions were detected in F-18-FDG (detection rate 97%).

The investigators conducted a comparative study of the clinical diagnostic efficacy of F-18-DHT and F-18-FDG in advanced prostate cancer (NCT00588185), using computational means to analyze the relationship between androgen receptor expression and the uptake of F-18-DHT, further confirmed its feasibility in clinical applications such as early diagnosis of prostate tumors. In 2014, the research group launched the study of using F-18-DHT as a molecular probe to simultaneously diagnose prostate cancer with PET and MR, and promoted the clinical application of F-18-DHT probe in the diagnosis and treatment of prostate tumor hormones.

A novel molecular probe PSMA targeting: with the deepening of molecular biology research, PSMA receptor becomes an ideal targeting for molecular contrast and prostate cancer treatment. It depends on the pharmaceuticals including monoclonal antibodies, peptides, small molecules and PSMA sites of action, the PSMA targeting antibodies are categorized into intracellular domain antibodies, such as 7EI1 and PM2J004.5, and extracellular domain antibodies, such as J591, J415, and PEQ226.6 and so on. In the above-mentioned methods including F-18-FDG, some cases cannot judge prostate cancer, especially in the case of metastasis. The current study found that prostate cancer cells surface highly expressed PSMA, so as long as anti-PSMA can be developed, the relevant radiopharmaceuticals can be developed as well. A variety of antibodies or peptides (J591, PSMA-11, etc.) have been developed and can be diagnosed using the Ga-68 labeling. However, clinical progress has moved toward the concept of PRRT (peptide receptor radionuclide therapy), and it is hoped that the diagnosis and treatment will move forward in synchronization.

At present, there are quite a few kinds of classes developed on the basis of PSMA, mainly for diagnostic use. Only the improved PSMA-617 can be labeled with Lu-177 for treatment. The pharmaceutical has now entered clinical phase III, expected to be completed by 2020, proving that effective treatment can be achieved. However, since the half-life of the pharmaceutical is 10.8 hours, the patient needs about 4 times in the overall course of treatment, which consumes more time and money.

The prior art US Publication No. US2016/0228587 A1 discloses a functional linker which allows a prostate cancer suppressing agent to bind to a chelating agent and chelate with a radioactive nucleus to be traced and diagnosed as labeling for prostate cancer, but the pharmaceutical has a short half-life and multiple treatments is in need.

The difference between the present invention and the foregoing prior art is that the compound disclosed in US2016/0228587 A1 is modified by Evans Blue to increase the function of the compound to bind to blood albumin, and to effectively reduce the shortcomings of compound excretion through the kidney after entering the living body, and reduces the dosage of the pharmaceutical by more than 75%.

In addition, the prior art Chinese Patent Publication No. CN104650217 A discloses the use of Evans Blue or Evans Blue derivative modified Exendin-4 for the treatment of Type II diabetes and myocardial infarction, although the same use of Evans Blue or Evans Blue modified compound in accordance with the present invention, however, the patent application CN 104650217 A discloses that the modified form and function are completely different. The present invention provides a linker which can be combined with a chelating agent and a truncated Evans blue, which can label radioactive species Ga-67, Ga-68, In-111, Lu-177, Cu-64, and Y-90 for use in analysis and evaluation of human prostate cancer tumor model images.

The PSMA-7165 derivative provided by the present invention has high binding capability to the PSMA receptor, and is modified based on the above PSMA-617 and the truncated Evans Blue is added by using a linker to increase the half-life in the body to prolong its residence time in the blood and achieve the goal of treatment with a single injection.

### SUMMARY OF THE INVENTION

The main object of the present invention is to provide a peptide derivative of PSMA (prostate-specific membrane antigen, PSMA) targeting radiotherapy, which is based on PSMA-617 and synthesizes a PSMA-7165 diagnostic tracer with radiolabeling function, and uses the PSMA-7165 derivative. Highly binding to PSMA receptors, and PSMA receptors are expressed on prostate cancer, which can be used as a target diagnostic or therapeutic pharmaceutical for prostate cancer. Based on PSMA-617, Evans Blue was added to prolong its residence time in the blood and increase the half-life in the body. It can achieve the goal of complete treatment by a single injection, thereby reducing the cost of time and money for patients.

Another object of the present invention is to provide a peptide derivative of PSMA targeting radioactive diagnosis and treatment, wherein the PSMA-7165 label is convenient and time-reduced, and the labeling efficiency can be more than 95% without being marked and purified by other columns.

From the experimental data of tumor animals, it is known that it has high binding to tumors. In the prostate cancer animal model (PSMA+), it is only 2 to 4 hours to see the obvious tumor accumulation images, and the high accumulation volume is maintained until 72 hours.

Another object of the present invention is to provide a peptide derivative of PSMA targeting radiotherapy, which can be administered by radioactive injection mode after the PSMA-7165 peptide derivative is labeled, or can be administered by a combination of frozen crystals. The convenience of transportation and the selectivity of back-end use increase the promotion of the pharmaceutical market. The peptide derivative of targeting radiotherapy of the present invention provides the Ga-67 or In-111 labeled PSMA-7165 radiopharmaceutical can be used for positron diagnosis of prostate cancers that experienced with low differentiation, metastasis and hormone therapy failure.

Other objects, advantages and novel features of the invention will become more apparent from the following detailed description when taken in conjunction with the accompanying drawings.

### BRIEF DESCRIPTION OF THE DRAWINGS

FIG. 1 is a diagram showing the chemical structure and function of the PSMA-7165 of the present invention including molecular weight;

FIG. 2 is a flow chart of labeling Ga-68-PSMA-7165 of the present invention.

FIG. 3 is a graph showing the efficiency and labeling efficiency of the Ga-68-PSMA-7165 of the present invention.

FIGS. 4a and 4b show the results of Radio-ITLC (Instant Thin-Layer Chromatography) analysis of Ga-68-PSMA-7165 of the present invention at various PH values, respectively.

FIG. 5 is a flow chart showing the labeling of Ga-67-PSMA-7165 of the present invention.

FIG. 6 shows analysis of labeling efficiency and radiochemical purity of the Ga-67-PSMA-7165 of the present invention.

FIG. 7 is a graph showing the results of Radio-ITLC analysis of Ga-67-PSMA-7165 of the present invention.

FIG. 8 is a flow chart showing the labeling of In-111-PSMA-7165 of the present invention.

FIG. 9 is a graph showing the efficiency and labeling efficiency of the In-111-PSMA-7165 of the present invention.

FIG. 10 is a binding assay of the In-111-PSMA-7165 and LNCaP (PSMA+) or PC3 (PSMA−) prostate cancer cells of the present invention.

FIG. 11 is a chart showing the peptide concentration of PSMA-7165.

FIG. 12 is a NanoSPECT/CT angiogram of the In-111-PSMA-7165 of the present invention in the LNCaP human prostate cancer tumor animal model.

FIG. 13 is a flow chart showing the labeling of Ga-67-DOTA-EB.

FIG. 14 shows the Ga-67-DOTA-EB labeling efficiency and radiochemical purity analysis.

FIGS. 15a and 15b show the results of Radio-ITLC analysis of Ga-67-DOTA-EB at various PH values, respectively.

FIG. 16 is a flow chart of the In-111-DOTA-EB.

FIG. 17 shows In-111-DOTA-EB labeling and labeling efficiency analysis table.

FIG. 18 shows the results of Radio-ITLC analysis of In-111-DOTA-EB.

FIG. 19 is a diagram showing chemical structural formula of PSMA-7165 of the present invention.

### DETAILED DESCRIPTION OF THE PREFERRED EMBODIMENTS

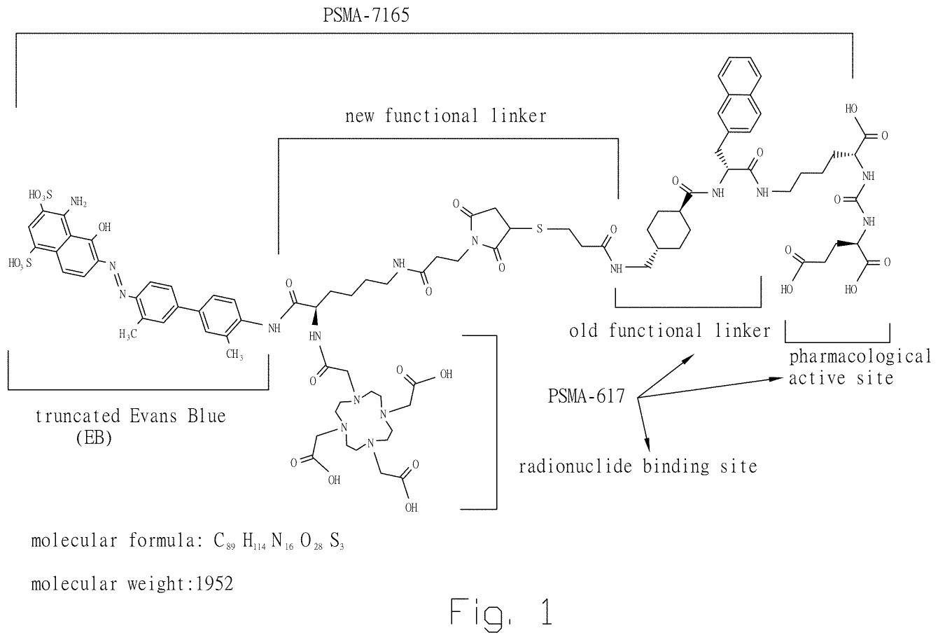

Referring to FIG. 1, the chemical structure of PSMA-7165 of the present invention is shown. In one preferred embodiment, the chemical synthesis of PSMA-7165 can be carried out via the disclosure of Schemes 1, 2, and 3 of the present invention as shown below.

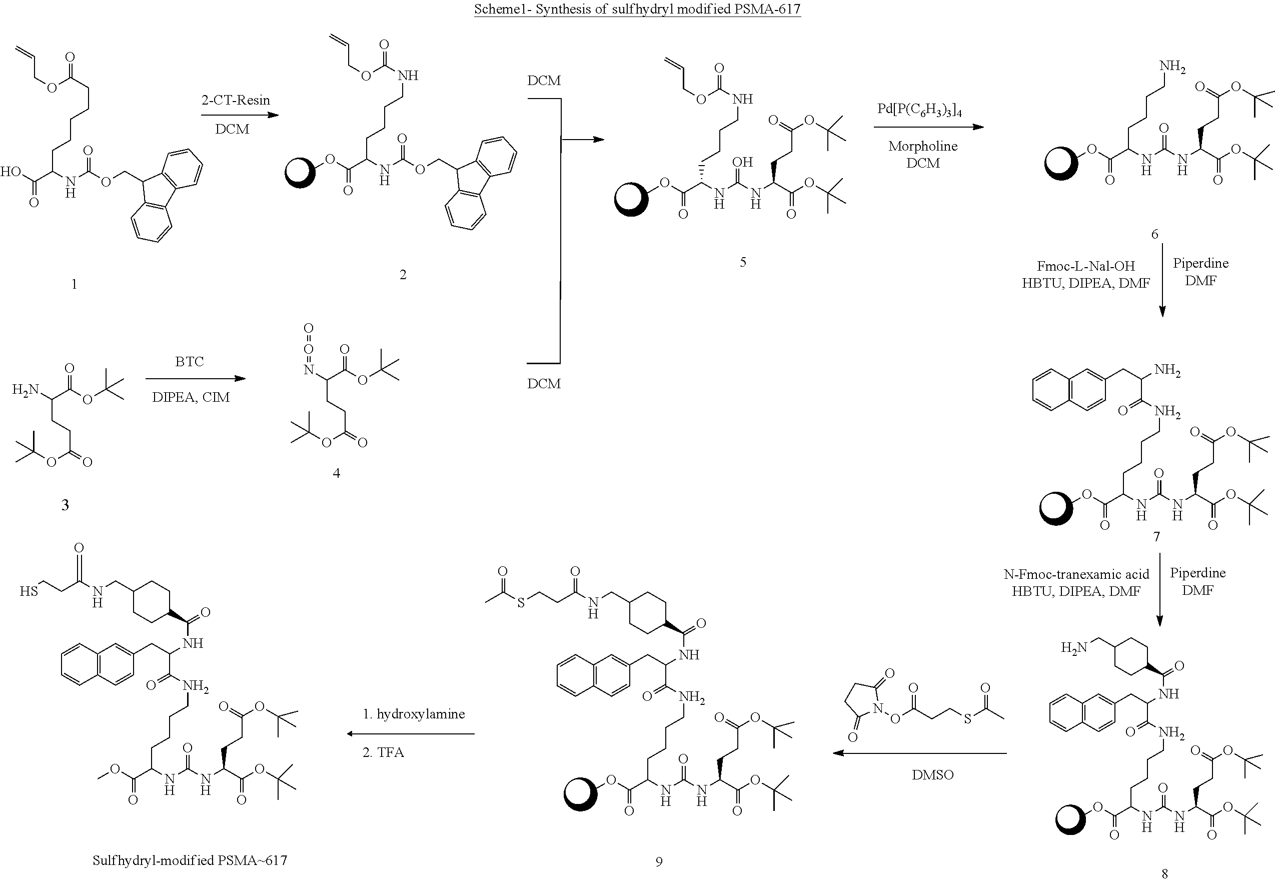

The chemical structure shown in the Scheme 1 comprises steps of:

placing a compound 1 of Boc glutamic acid in an ice bath of dichloromethane for 10 minutes, and adding a tri-phosgene for reaction through stirring at 0° C. for 6 hours to obtain an intermediate product isocyanate 2;

carrying out reaction by placing a compound 3 of ionic acid derivative and 2-chlorotrityl resin in ichloromethane at room temperature for 2 hours to obtain an intermediate product 4;

coupling intermediate product 2 and the intermediate product 4 through stirring at room temperature for 16 hours obtain an intermediate product 5;

placing the intermediate product 5, tetrapalladium (triphenylphosphine) and morpholine in dichloromethane, and stirring at room temperature for 3 hours to remove allyloxy protecting group to obtain an intermediate product 6;

stirring a mixture of Fmoc-3-(2-naphthyl)-L-alanine, HBTU (2-(1H-benzotriazol-1-yl)-1,1,3,3-tetramethyluronium hexafluorophosphate), DIPEA (N,N-Diisopropylethylamine) and the intermediate 6 at room temperature for 16 hours to obtain an intermediate product 7;

stirring tranexamic acid derivative, HBTU, DIPEA and the intermediate 7 at room temperature for 16 hours to obtain an intermediate product 8;

placing N-Succinimidyl-S-acetylthiopropionate (STPA) and sodium hydrogen phosphate in dimethyl hydrazine with the intermediate product 8 and stirring at room temperature for 10 hours to obtain an intermediate product 9, and

removing ethyl sulfhydryl group through carrying out reaction of the intermediate product 9 with hydroxylamine, followed by adding trifluoroacetic acid to obtain the first semi-finished product, naming sulfhydryl-modified PSMA-617, a PSMA-7165 intermediate.

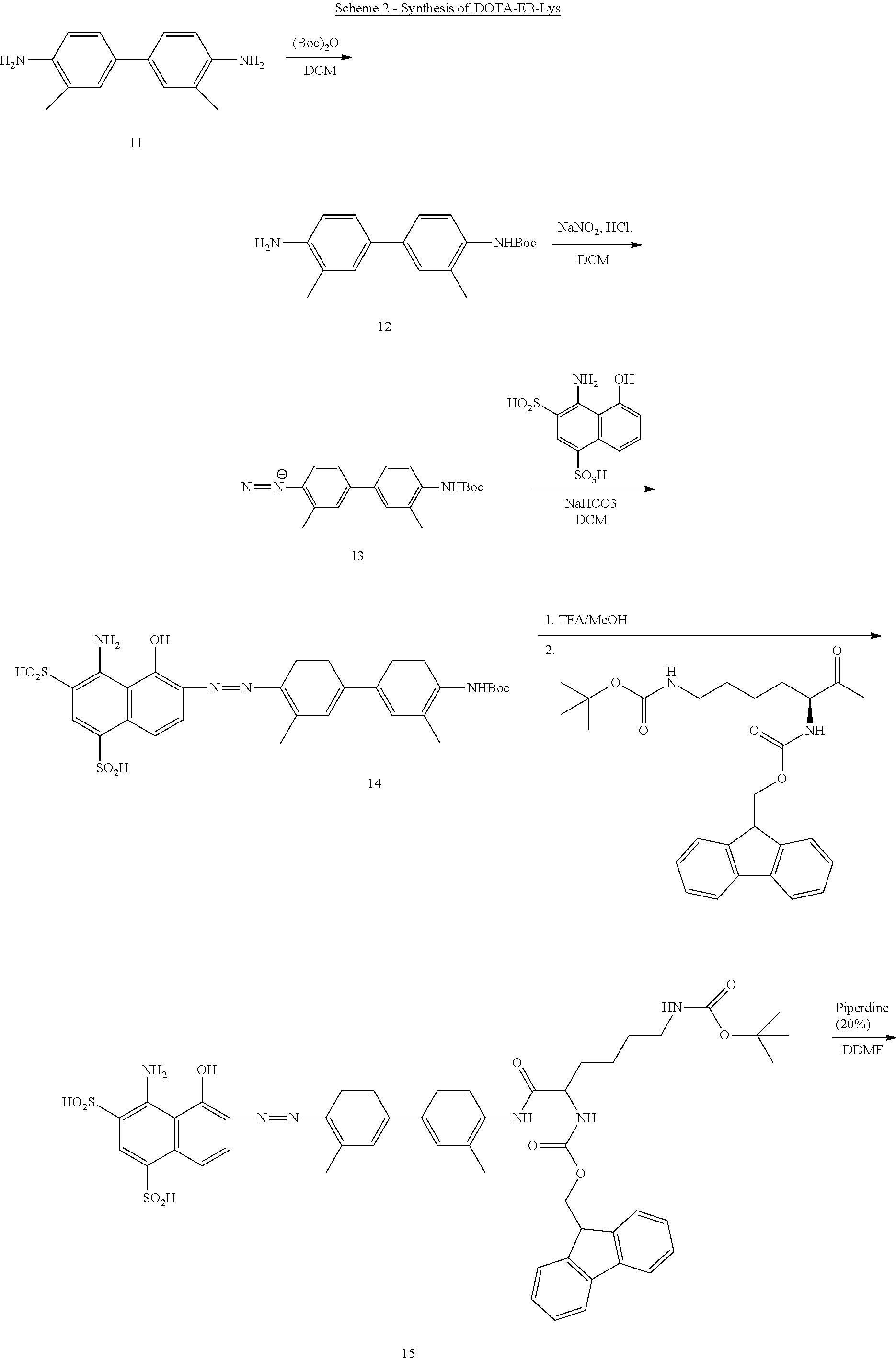

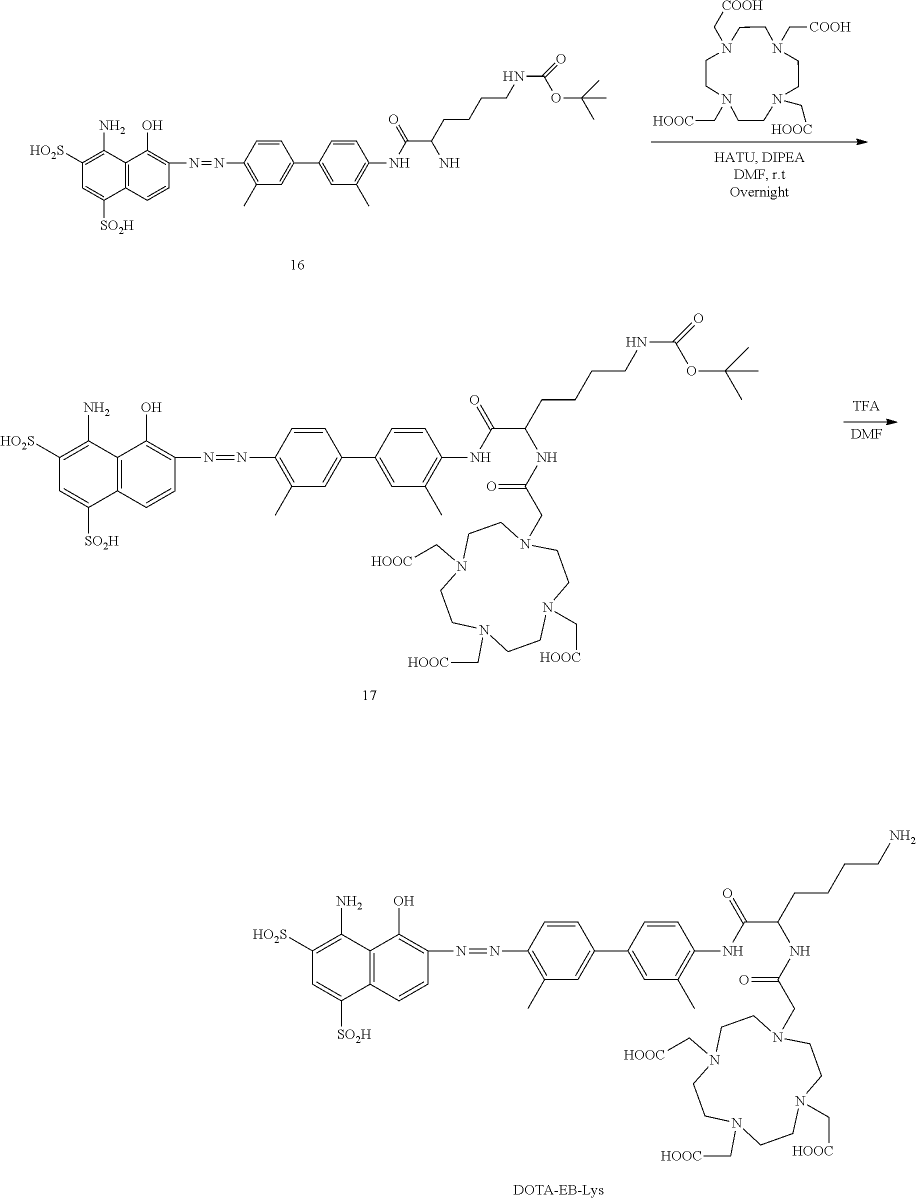

The chemical structure shown in the Scheme 2 comprises steps of:

carrying out reaction of compounds 11 (o-dimethylbenzidine) and di-tert-butyl dicarbonate in dichloromethane at room temperature for 5 hours, and using di-tert-butyl dicarbonate as a limited reagent, and carry out reaction with sodium nitrite and hydrochloric acid to form a diazonium salt intermediate 13 and obtain an intermediate product 12 (Boc-benzidine);

dissolving sodium 1-amino-8-naphthol-2,4-disulfonate and sodium hydrogencarbonate in water, and adding the intermediate product 13 slowly to carry out reaction in the solution for 12 hours to obtain an intermediate product 14, followed by adding the trifluoroacetic acid to the intermediate product 14 to remove the boc protecting group, adding Boc-Lys-Fmoc and HATU and stirring at room temperature for 6 hours to obtain intermediate product 15, and adding the intermediate product 15 to piperidine and stirring at room temperature for 4 hours to obtain intermediate product 16;

after pre-stirring the NOTA, HATU, and DIPEA in DMF for 15 minutes, the intermediate product 16 is added for reaction at room temperature overnight to obtain the intermediate product 17, and

adding the intermediate product 17 to trifluoroacetic acid and DMF solvent and stirring at room temperature for 2 hours to obtain a second semi-finished product DOTA-EB-Lys, PSMA-7165 intermediate.

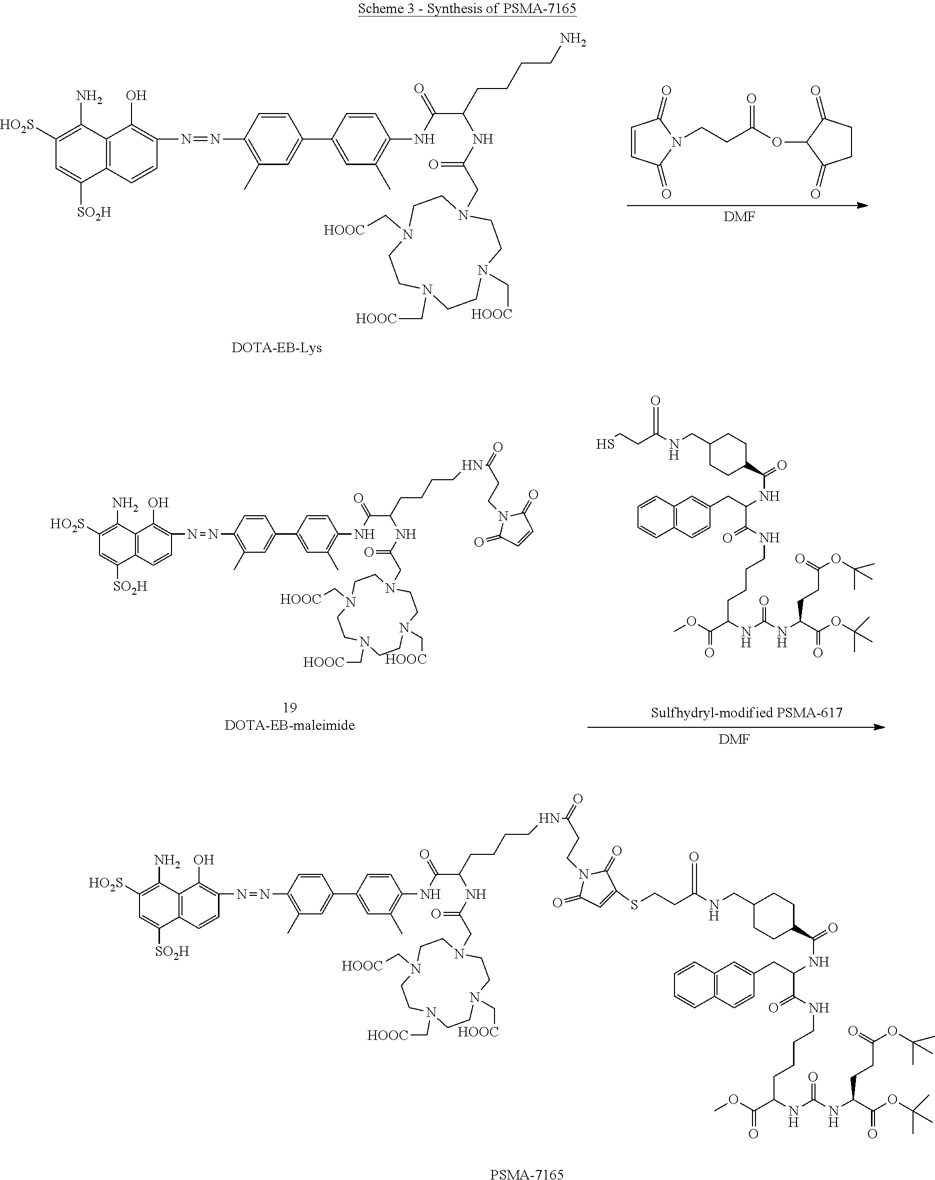

The chemical structure shown in the Scheme 3 comprises steps of:

stirring the second semi-finished product (DOTA-EB-Lys) and the dioxetane derivative at room temperature to obtain an intermediate product 19, and stirring the intermediate product 19 and the first semi-finished product (PSMA617-SH) in a PBS buffer solution and DMF at room temperature for 2 hours to obtain the final product PSMA-7165.

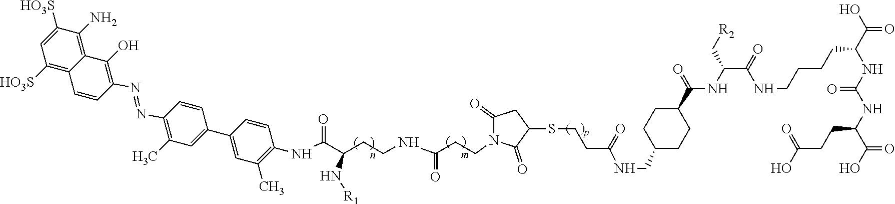

wherein n is an integer 1: m is an integer 1: p is an integer 1: R1 is a metal chelating group DOTA: R2 is a chemical structure of naphthalene molecule.

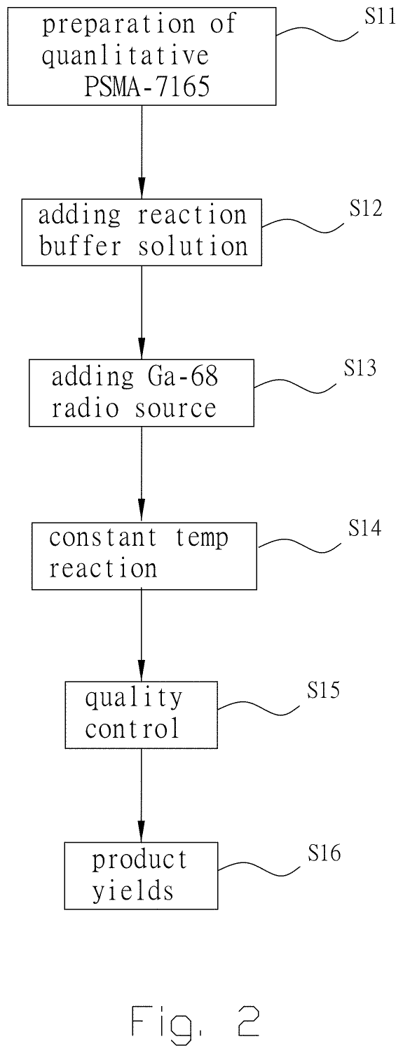

Referring to FIG. 2 to 4b, it can be seen that the preparation method of the Ga-68 radioactive labeling of PSMA-7165 of the present invention comprises: preparing the quantitative PSMA-7165 of S11 step, adding the reaction buffer of S12 step, adding the Ga-68 source of S13 step, constant temperature reaction of S14 step, quality control of S15 step, and finished product output of S16 step;

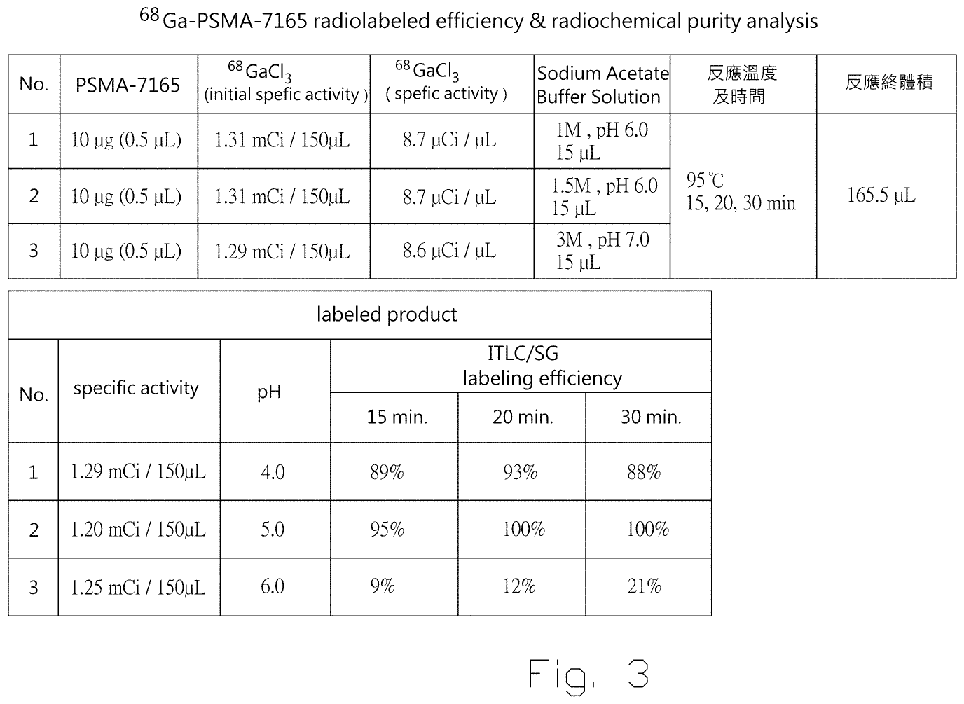

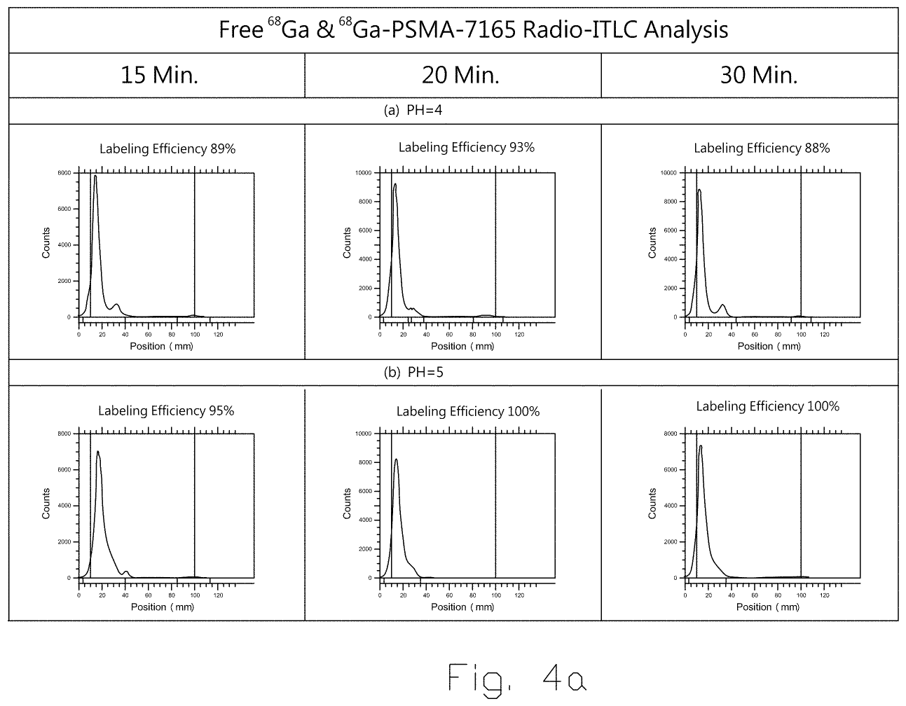

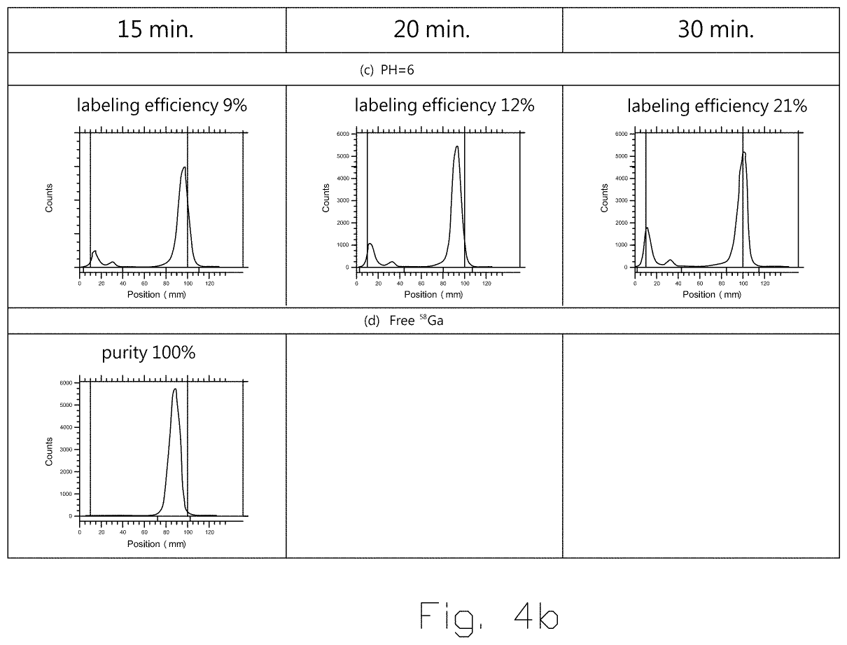

The S11 step is to add PSMA-7165 in DMSO (dimethyl sulfoxide) at 20 mg/mL and dispense in a 1.5 mL microcentrifuge tube at 10 μg, and store at −20° C., and the 1.5 mL microcentrifuge tube containing 10 μg of PSMA-7165 is thus prepared for labeling; the step S12 is to add 1.5 μg of PSMA-7165 in a 1.5 mL microcentrifuge tube to the 1, 1.5 or 3 M sodium acetate buffer solution at a pH of 6.0, 6.0 or 7.0, respectively, and the pH values of the reaction solutions are 4.0, 5.0 and 6.0, respectively; the S13 step is to perform an ultrasonic oscillation of the mixture obtained in the S12 step for 1 to 2 min. and add Ga-68 radioactive source with starting activity about 1.3 mCi, completely mixed in uniformity, and the final reaction volume obtained is 165.5 μL; the step S14 is to carry out heating process in a precision thermostat controller at 95° C. for 15, 20 or 30 minutes and the oscillation speed is 500 rpm during the heating process; the step S15 is, after completely cooling, to take an appropriate amount of sample for radio-ITLC (radioactive instant thin-layer chromatography) analysis, the development solution for analysis is 0.1M citric acid, in the analysis system, the origin is Ga-68-PSMA-7165 with radioactive labeling and the mobile end of the solution is without radioactive labeling Ga-68; the S16 step is to reach 95% labeling efficiency after 15 minutes labeling with a 1.5M sodium acetate buffer at pH 5.0 under different conditions, when the reaction time is increased to 20 or 30 minutes, the labeling efficiency can reach 100%. The labeling efficiency analysis is shown in FIG. 3 to 4b.

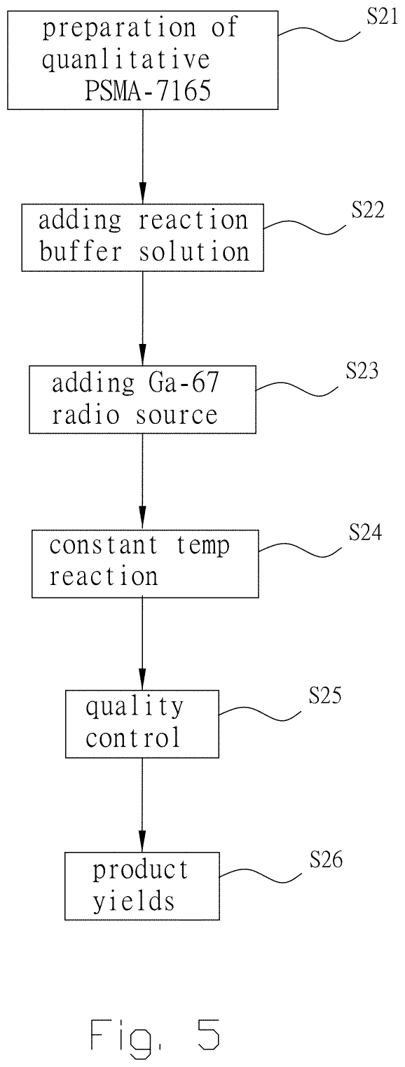

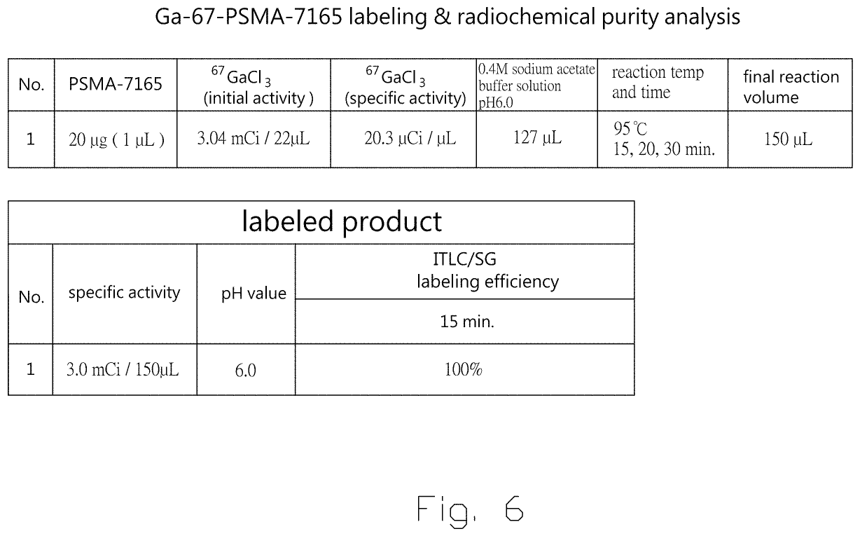

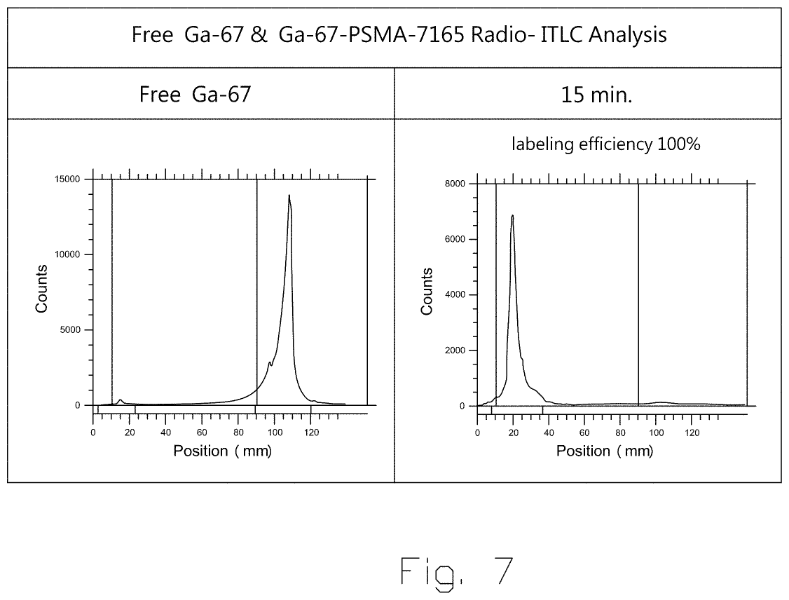

Referring to FIG. 5 to 7, it can be seen that the preparation method of the Ga-67 radioactive labeling of PSMA-7165 of the present invention, includes: preparing a quantitative PSMA-7165 of S21 step, adding a reaction buffer of S22 step, adding Ga-67 source of S23 step, constant temperature reaction of S24 step, quality control of S25 step, and finished product output of S26 step, in which, the S21 step is to add PSMA-7165 in DMSO at 20 mg/mL, dispense in a microcentrifuge tube at 20 μg, store at −20° C., and 20 μg of PSMA-7165 is taken out from the 1.5 mL microcentrifuge tube for labeling; the step S22 is to add a 0.4 M sodium acetate buffer solution having a pH 6.0; the S23 step is to perform an ultrasonic oscillation with the mixture obtained in the S22 step for 1 to 2 min., then add Ga-67 source with starting activity at about 3 mCi, completely mixed in uniformity, to obtain the final reaction volume of 150 μL; the step S24 is to carry out in a precision thermostat controller at 95° C. for 15, 20 or 30 minutes with the oscillation speed 500 rpm at the same time during the heating process; the S25 step is, after being completely cooled, to take an appropriate amount of sample for radio-ITLC (radioactive instant thin-layer chromatography) analysis, the analysis development solution is 0.1 M citric acid, in this analysis system, the origin is of labeling with radioactive Ga-67-PSMA-7165 and the mobile end of the solution is without radioactive labeling Ga-67; the step S26 is to label with a 0.4M sodium acetate buffer solution at pH 6.0 for 15 minutes to obtain a labeling efficiency 100%, and the labeling efficiency analysis is shown in FIG. 6 to 7.

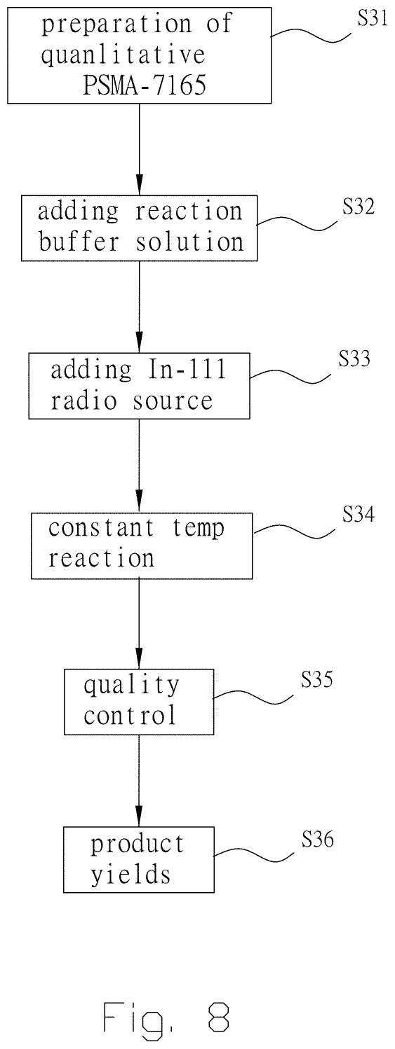

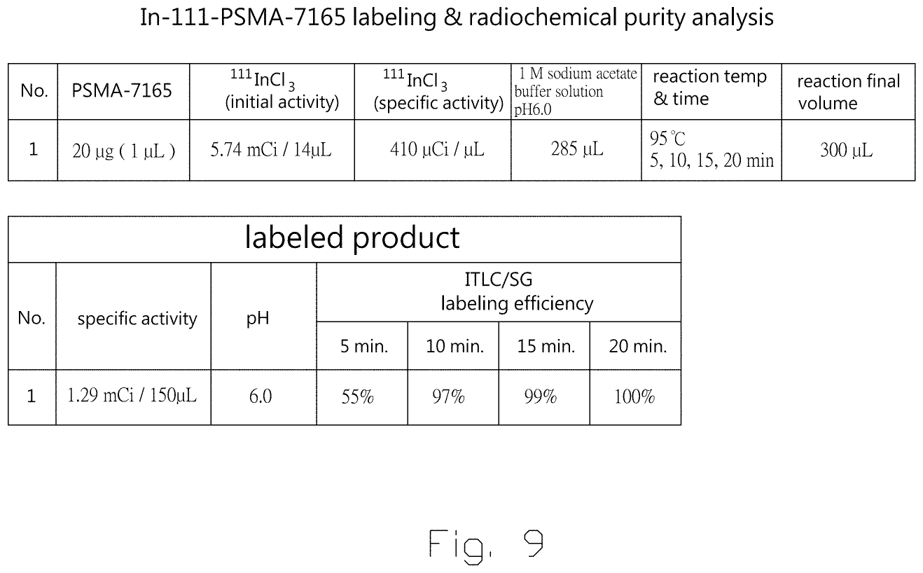

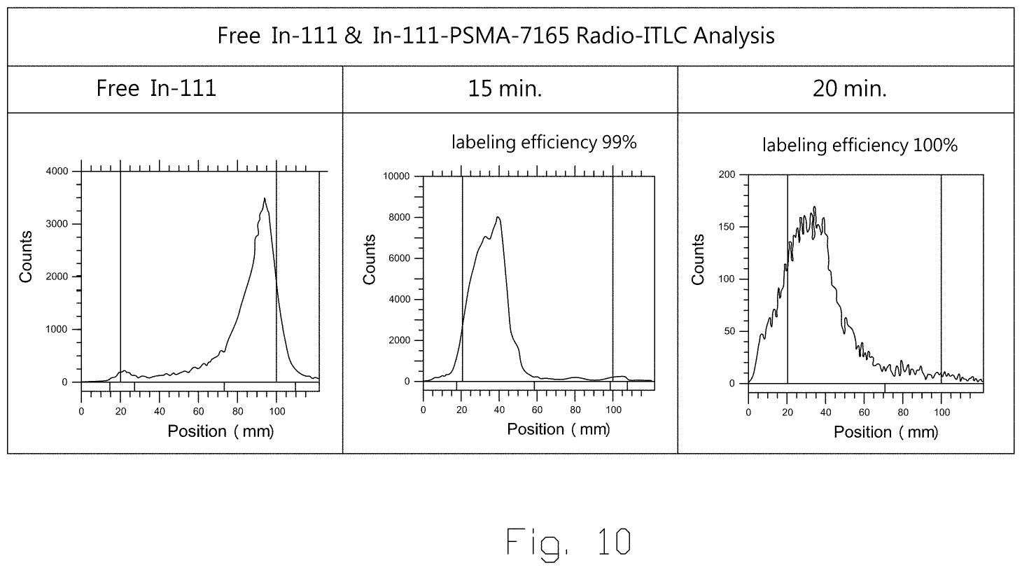

Referring to FIG. 8 to 10, it can be seen that the method for preparing the In-111 radioactive label of the PSMA-7165 of the present invention comprises the steps of: preparing a quantitative PSMA-7165 of S31 step, adding a reaction buffer of S32 step, add In-111 radioactive source of S33 step, constant temperature reaction of S34 step, quality control of S35 step, and finished product output of S36 step, in which, the S31 step is to add PSMA-7165 in DMSO at 20 mg/mL, dispense in a microcentrifuge tube at 20 μg, to store at −20° C., and 20 μg of PSMA-7165 is taken out from the 1.5 mL microcentrifuge tube for labeling; the S32 step is to add a 1 M sodium acetate buffer solution at pH 6.0; the step S33 is to perform an ultrasonic oscillation with the mixture obtained in the S32 step for 1-2 min, and then add the In-11 radioactive source at an initial activity about 5.7 mCi, completely mixed in uniformity, the final reaction volume of 300 μL is obtained; the step S34 is to carry out in a precision thermostat controller at 95° C. for 5, 10, 15 or 20 minutes, and the oscillation speed is 500 rpm during the heating process; the step S35 is to, after completely cooling, take an appropriate amount of sample for radio-ITLC (radioactive thin-layer chromatography) analysis, the analysis using developing solution 0.1M citric acid and 0.1M sodium citrate, and is administered in 2 to 8 volume ratio configuration, in this analysis system, the origin is the labeled In-111-PSMA-7165 and the mobile end is the solution without radioactive labeling In-111; the step S36 is to label with a 1M sodium acetate buffer solution at pH 6.0 and the labeling efficiency can reach 97% after 10 minutes. The labeling efficiency analysis is shown in FIG. 9-10.

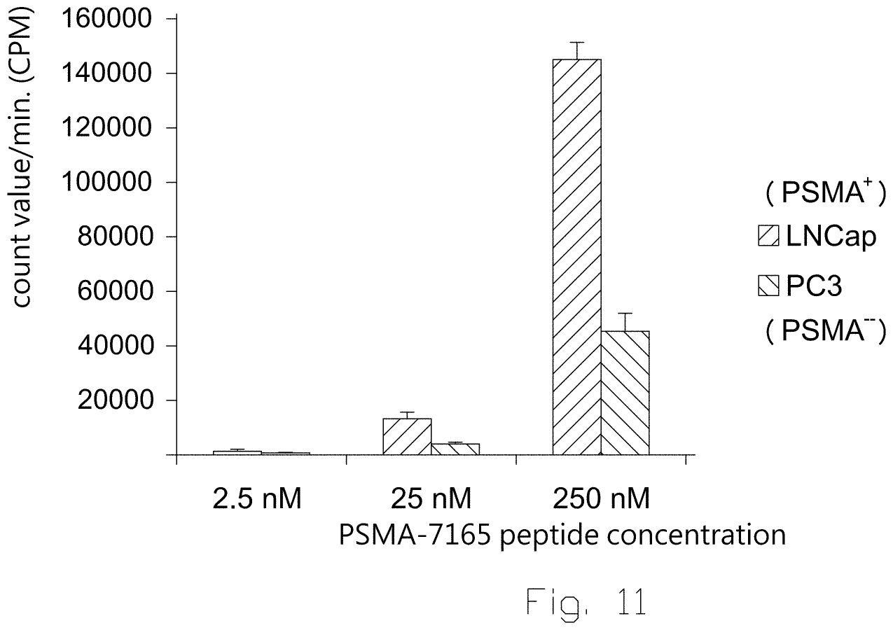

Referring to FIG. 11, it can be seen that in the in vitro cell binding activity test of In-111-PSMA-7165, the LNCaP cells exhibiting PSMA and the PC3 cells not expressing PSMA are cultured from 7×105 cells with different peptide concentration of In-111-PSMA-7165 at 37° C. for 45 minutes, and then the cell-bound In-111-PSMA-7165 was washed with ice PBS (phosphate buffered saline), and the precipitated cells were collected through centrifugation. The radionuclide readings were read by gamma-counter and the LNCaP cell lines expressing PSMA at different peptide concentrations of 2.5 nM, 25 nM or 250 nM had higher CPM (count per minute) readings.

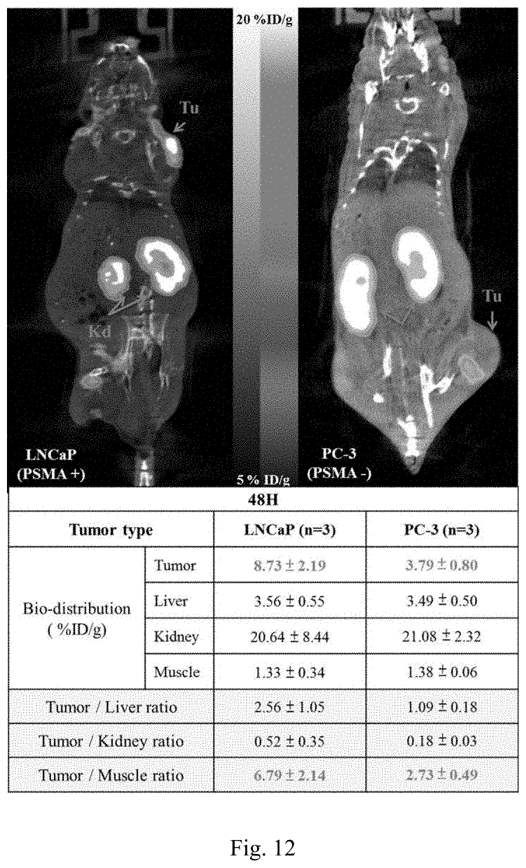

Referring to FIG. 12, it can be seen that the PSMA-7165 labeled with In-111 of the present invention has presented a pharmaceutical distribution over a period of 48 hours in an LNCaP (PSMA+) and PC3 (PSMA−) for mouse prostate tumor contrast test. PSMA-expressing LNCaP human prostate cancer cells 4×10⁶ were inoculated in the right forelimb of SCID (severe combined immunodeficiency) mice or PC-3 human prostate cancer cells 2×10⁶ not expressing PSMA were inoculated to the hind limbs of BALB/c (Bagg Albino, inbred research mouse strain) nude mice, and about 3 weeks after the animals were inoculated, PSMA-7165 was labeled with the radioisotope In-11, and then the radiolabeling analysis confirmed that the pharmaceutical was 100% efficient. The labeled finished product was adjusted to about 500 μCi/100 μL per injection needle with injection water, and Nano-SPECT/CT angiography was performed after 1, 4, 24, and 48 hours of administration of In-111-PSMA-7165 by tail vein injection.

The NanoSPECT/CT angiography and image semi-quantitative steps include: (1) Injecting a known activity of In-111-PSMA-7165 pharmaceutical into LNCaP tumor mice by tail vein injection, (2) Prepare a fixed-activity prosthesis as a reference substance, and fix it on the bed frame of the NanoSPECT/CT instrument together with the tumor mouse. During the contrast, the tumor mice receives the animal medication Aining anesthetic (Attane, provided by Panion & BF Bio-Tech Inc.) for live anesthesia, (3) Perform NanoSPECT/CT angiography at different time points according to the experimental design, using the Nucline (V.2.00) software developed by Mediso Inc., and SPECT: 60 sec/frame, Energy Window 245±10% keV&171±10% keV was used for angiography and image reconstruction was performed using HiSPECT (V.1.43049) image reconstruction software developed by Scivis Inc, (4) Reconstructed images used image analysis software InVivoScope (V.1.44) developed by Mediso Inc. for preliminary inspection of SPECT and CT images and production of image files, and (5) Semi-quantitative analysis of images using PMod (V.3.4) image analysis software developed by PMOD Technologies, using the SPECT count value obtained from the reference material of known activity at each time point, and the linear ratio formula between the SPECT count value and the actual activity is derived, and the SPECT count value obtained by each circled target organ, and the activity/volume of each target organ is obtained by interpolation or extrapolation of a linear formula, assuming 1 g per 1 mL of the injection and 1 g per 1 cm³ of the circled area in the mouse, and it is possible to deduce the organism distribution ratio (% ID/g) by each gram of pharmaceutical in each organ by means of the known activity volume of the administered injection and the weight of the experimental mouse.

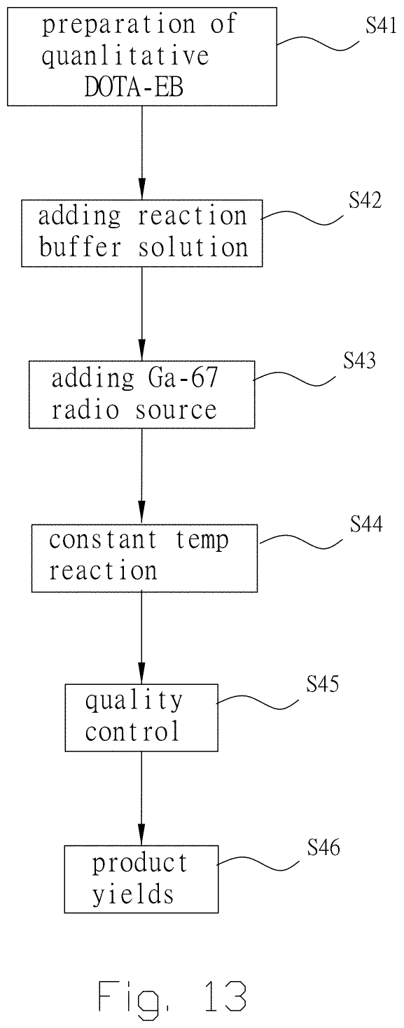

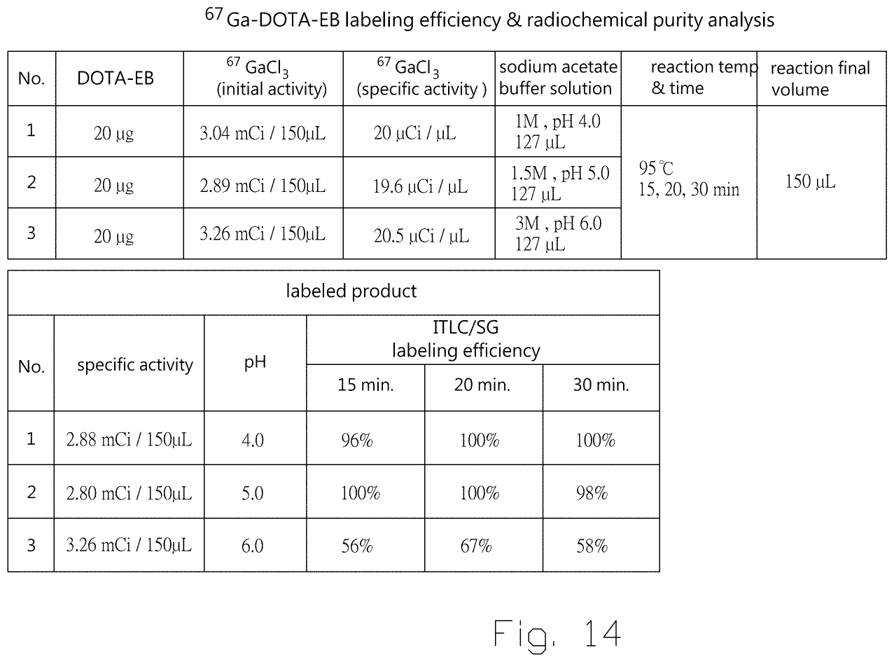

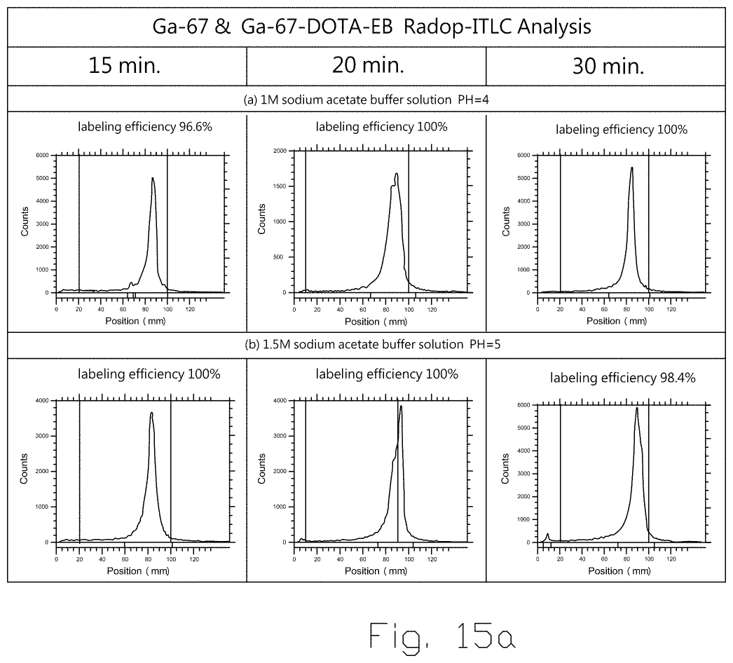

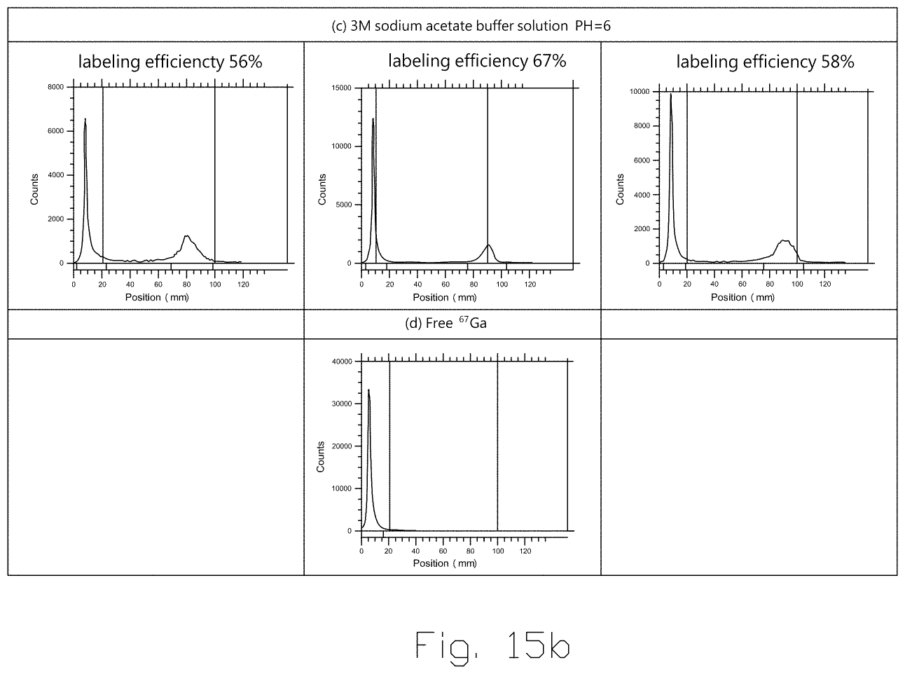

Referring to FIG. 13 to 15b, it can be seen that the preparation method of the DOTA-EB Ga-67 radioactive label of the present invention comprises: preparation of quantitative DOTA-EB of S41 step, add reaction buffer of S42 step, add Ga-67 radioactive source of S43 step, constant temperature reaction of S44 step, quality control of S45 step, and finished product of S46 step; the S41 step is to prepare DOTA-EB in DMSO at 20 mg/mL, and place in a microcentrifuge tube at 20 μg, store at −20° C., and 20 μg of DOTA-EB is taken out from the 1.5 mL microcentrifuge tube when labeling is to be performed, the S42 step is to add 1, 1.5, 3M sodium acetate solution reaction buffer solution at pH 6.0, 6.0 or 7.0, respectively, so that the final reaction solution pH value is 4.0, 5.0 or 6.0, respectively, and S43 step is to perform an ultrasonic oscillation with the mixture obtained in the S42 step for 1 to 2 min, and then add the Ga-67 radioactive source at an initial activity about 3 mCi, completely mixed in uniformity, and the final reaction volume of 150 μL is obtained; the step S44 is to carry out in a precision thermostat controller at 95° C. for 5, 10, 15 or 20 minutes, and the oscillation speed is 500 rpm during the heating process; the step S45 is to, after completely cooling, take an appropriate amount of sample for radioactive thin layer analysis radio-ITLC, the analysis of the development solution is 10% methanol (MeOH), in this analysis system, the origin is unreacted Ga-67 and the mobile end is the solution Ga-67-DOTA-EB with labeled radioactivity; the product yield of step S46 is to label with a 1M sodium acetate buffer solution at pH 4.0 or 1.5M sodium acetate solution at pH 5.0, and the labeling efficiency can reach more than 95% after 15 minutes of labeling, and if the reaction time is increased to 20 or 30 minutes, the labeling efficiency is still above 95%, and the labeling efficiency analysis is shown in FIG. 14 to 15b.

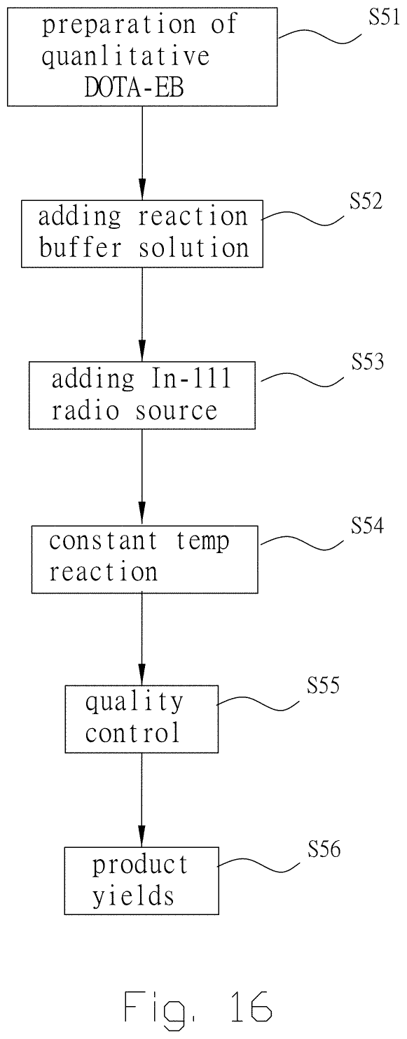

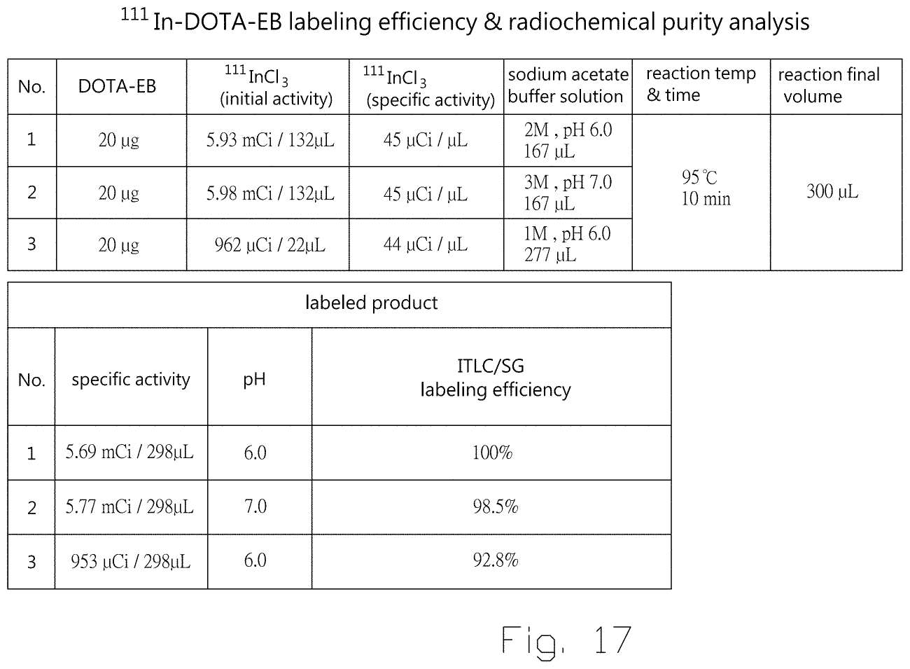

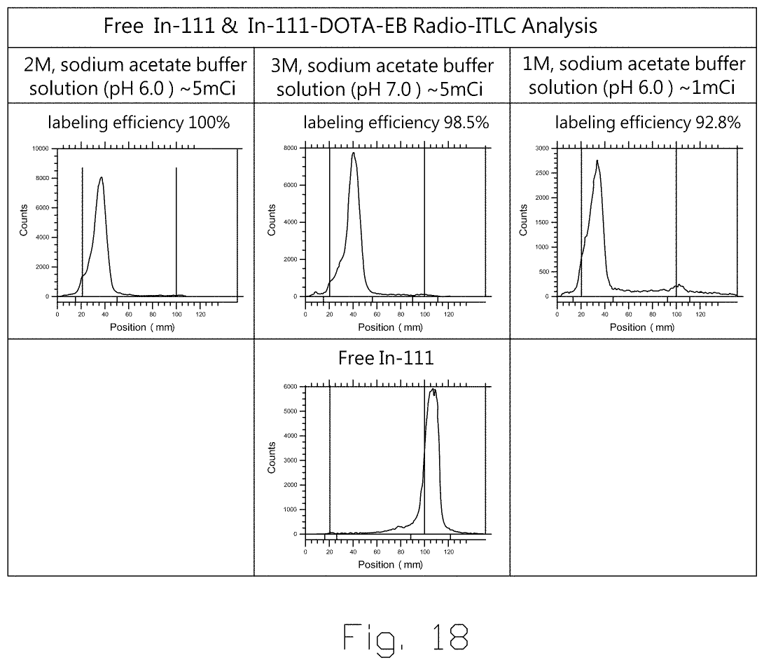

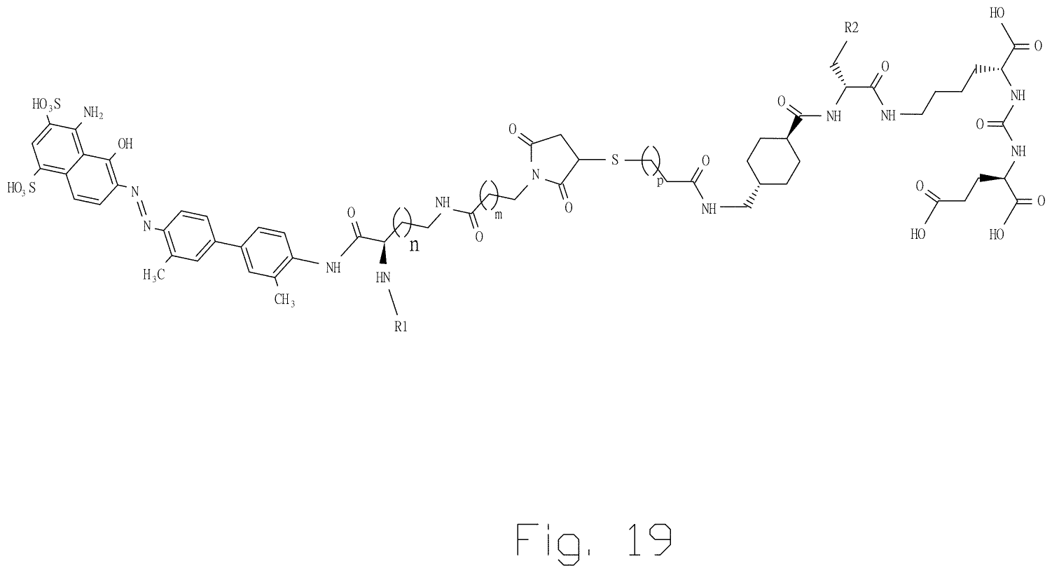

Referring to FIG. 16 to 18, it can be seen that the preparation method of the DOTA-EB In-111 radioactive labeling of the present invention comprises the steps of: preparing quantitative DOTA-EB of S51 step, adding reaction buffer of S52 step, adding In-111 radioactive source of S53 step, constant temperature reaction of S54 step, quality control of S55 step, and finished product yield of S56 step, in which, the S51 step is to prepare DOTA-EB in DMSO at 20 mg/mL, place in a microcentrifuge tube at 20 μg, store at −20° C., and 20 μg of DOTA-EB is taken out from the 1.5 mL microcentrifuge tube when labeling is to be performed, the step S52 is to add 1, 2 or 3 M sodium acetate solution, the pH values of which are 6.0, 6.0 or 7.0, respectively, and the pH values of the final reaction solution are 6.0, 6.0 and 7.0, respectively; the S53 step is to perform an ultrasonic oscillation with the mixture obtained from the step S52 for 1 to 2 min., then add In-111 radioactive source with starting activity about 1 or 5 mCi, completely mixed in uniformity, and the final reaction volume of 300 μL is obtained; the step S54 is to carry out in a precision thermostat controller at 95° C. for 10 minutes and oscillation speed is 500 rpm during the heating process; the S55 step is, after completely cooling, to take an appropriate amount of sample for radioactive thin layer analysis radio-ITLC, the analysis using the developing solution 0.1M citric acid and 0.1 M sodium citrate, and is administered in 2 to 8 volume ratio configuration, in this analysis system, In-111-DOTA-EB is located at the origin, and unreacted In-111 is located at the developing liquid end; the S56 step is in different conditions, the efficiency of the reaction labeling can reach more than 90% after 10 minutes of labeling, and the labeling efficiency analysis is shown in FIG. 17-18.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A peptide derivative produced for targeting radiotherapy in accordance with the method of claim 20, comprising a nuclear portion R1, a truncated Evans blue, a new functional connector, an old functional connector in a pharmacologically active structure, which interacts with radioisotopes forming radioactive peptides for diagnostic and therapeutic, is shown below: 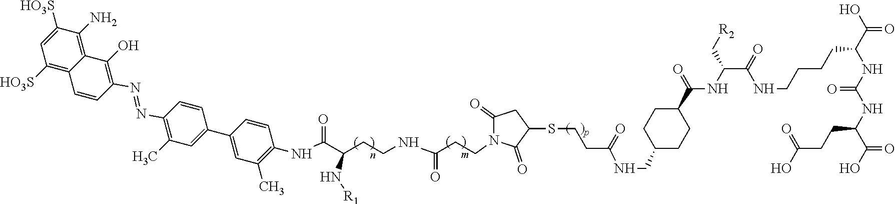
wherein n is an integer 1-12:
m is an integer 1-5:
p is an integer 1-5:
R1 is a metal chelating group DOTA:
R2 is a chemical structure of naphthalene molecule.

2. The peptide derivative for targeting radiotherapy according to claim 1, wherein the nuclear moiety comprises a metal chelate compound DOTA, NOTA or DTPA, which is used in labeling with radioisotopes.

3. The peptide derivative for the targeting radiotherapy according to claim 1, wherein the new functional connector has an integer of 1 to 12 in the parameter n.

4. The peptide derivative for the targeting radiotherapy according to claim 1, wherein the old functional connector has a parameter R2 naphthalene.

5. The peptide derivative of the targeting radiotherapy according to claim 1, wherein the radioisotope is ⁶⁸Ga, ⁶⁷Ga, ¹¹¹In, ¹⁷⁷Lu, ⁶⁴C or ⁹⁰Y is used for diagnosis or treatment in radiotherapy.

6. The peptide derivative for the targeting radiotherapy according to claim 1, wherein the targeting radiotherapy peptide derivative PSMA-7165 is combined with a labeling radioisotope Ga-68 which is used for positron diagnosis.

7. The peptide derivative for the targeting radiotherapy according to claim 1, wherein the targeting radiotherapy peptide derivative PSMA-7165 is combined with a labeling radioisotope Ga-67 which is used for single photon diagnosis.

8. The peptide derivative for the targeting radiotherapy according to claim 1, wherein the targeting radiotherapy peptide derivative PSMA-7165 is combined with a labeling radioisotope In-111 which is used for single photon diagnosis or treatment and animal organism distribution test.

9. The peptide derivative for the targeting radiotherapy according to claim 1, wherein the target diagnostic peptide derivative PSMA-7165 is combined with a labeling radioisotope Lu-177 which is used in animal testing and in vivo radiotherapy.

10. The peptide derivative for the targeting radiotherapy according to claim 1, wherein the targeting therapeutic peptide derivative PSMA-7165 is combined with sodium acetate salt, mannitol, gentisic acid or ascorbic acid for preparation of non-radioactive frozen crystal.

11. The peptide derivative for the targeting radiotherapy according to claim 10, wherein the non-radioactive frozen crystal preparation which is used for the radioactive diagnosis and treatment with Ga-68, Ga-67, In-111 or Lu-177 radioisotope in reaction to produce radiopharmaceuticals.

12. The peptide derivative for the targeting radiotherapy according to claim 10, wherein the non-radioactive frozen crystal preparation is a radiopharmaceutical labeling kit.

13. The peptide derivative for the targeting radiotherapy according to claim 12 wherein the radiopharmaceutical labeling kit is suitable for automation.

14. The peptide derivative for the target radioactive diagnosis and treatment according to claim 12, wherein the non-radioactive frozen crystal preparation is used to produce radioactive injection pharmaceutical through combination with Ga-68 in purification process.

15. The peptide derivative for the targeting radiotherapy according to claim 6, wherein the Ga-68 labeled PSMA-7165 is used for the positron diagnosis of prostate cancer experiencing low differentiation, metastasis and hormone therapy failure.

16. The peptide derivative for the targeting radiotherapy according to claim 7 wherein the Ga-67 or In-111 labeled PSMA-7165 is used for the positron diagnosis of prostate cancer experiencing low differentiation, metastasis and hormone therapy failure.

17. The peptide derivative for the targeting radiotherapy according to claim 8 wherein the Ga-67 or In-111 labeled PSMA-7165 is used for the positron diagnosis of prostate cancer experiencing low differentiation, metastasis and hormone therapy failure.

18. The peptide derivative for the targeting radiotherapy according to claim 8 wherein the In-111 labeled PSMA-7165 high activity pharmaceutical is used for in vivo radiotherapy of prostate cancer experiencing low differentiation, metastasis and hormone therapy failure.

19. The peptide derivative for the targeting radiotherapy according to claim 9 wherein the In-177 labeled PSMA-7165 is used for in vivo radiotherapy of prostate cancer experiencing low differentiation, metastasis and hormone therapy failure.

20. A method for preparing a peptide derivative for targeting radiotherapy, comprising first, second and third processes, wherein the first process comprises the steps of:
placing a compound 1 of Boc glutamic acid in an ice bath of dichloromethane for 10 minutes, and adding a tri-phosgene for reaction through stirring at 0° C. for 6 hours to obtain an intermediate product isocyanate 2;
carrying out reaction by placing a compound 3 of ionic acid derivative and 2-chlorotrityl resin in ichloromethane at room temperature for 2 hours to obtain an intermediate product 4;
coupling intermediate product 2 and the intermediate product 4 through stirring at room temperature for 16 hours obtain an intermediate product 5;
placing the intermediate product 5, tetrapalladium (triphenylphosphine) and morpholine in dichloromethane, and stirring at room temperature for 3 hours to remove allyloxy protecting group to obtain an intermediate product 6;
stirring a mixture of fluorene-acyl-chloro-3-(2-naphthalene)-L-isoamine, HBTU, DIPEA and the intermediate 6 at room temperature for 16 hours to obtain an intermediate product 7;
stirring tranexamic acid derivative, HBTU, DIPEA and the intermediate 7 at room temperature for 16 hours to obtain an intermediate product 8;
placing N-Succinimidyl-S-acetylthiopropionate (STPA) and sodium hydrogen phosphate in dimethyl hydrazine with the intermediate product 8 and stirring at room temperature for 10 hours to obtain an intermediate product 9;
removing ethyl sulfhydryl group through carrying out reaction of the intermediate product 9 with hydroxylamine, followed by adding trifluoroacetic acid to obtain the first semi-finished product, naming sulfhydryl-modified PSMA-617, a PSMA-7165 intermediate;
the second process comprises the steps of:
carrying out reaction of compounds 11 (o-dimethylbenzidine) and di-tert-butyl dicarbonate in dichloromethane at room temperature for 5 hours, and using di-tert-butyl dicarbonate as a limited reagent, and carry out reaction with sodium nitrite and hydrochloric acid to form a diazonium salt intermediate 13 and obtain an intermediate product 12 (Boc-benzidine);
dissolving sodium 1-amino-8-naphthol-2,4-disulfonate and sodium hydrogencarbonate in water, and adding the intermediate product 13 slowly to carry out reaction in the solution for 12 hours to obtain an intermediate product 14, followed by adding the trifluoroacetic acid to the intermediate product 14 to remove the boc protecting group, adding Boc-Lys-Fmoc and HATU and stirring at room temperature for 6 hours to obtain intermediate product 15, and adding the intermediate product 15 to piperidine and stirring at room temperature for 4 hours to obtain intermediate product 16;
after pre-stirring the NOTA, HATU, and DIPEA in DMF for 15 minutes, the intermediate product 16 is added for reaction at room temperature overnight to obtain the intermediate product 17;
adding the intermediate product 17 to trifluoroacetic acid and DMF solvent and stirring at room temperature for 2 hours to obtain a second semi-finished product (DOTA-EB-Lys, PSMA-7165 intermediate);
the third process comprises the steps of:
stirring the second semi-finished product (DOTA-EB-Lys) and the dioxetane derivative at room temperature to obtain an intermediate product 19, and stirring the intermediate product 19 and the first semi-finished product (PSMA617-SH) in a PBS buffer solution and DMF at room temperature for 2 hours to obtain the final product PSMA-7165.
